(19) United States
(12) Patent Application Publication
(10) Pub. No.: US 2008/0215050 A1
(43) Pub. Date: Sep. 4, 2008

(54) TISSUE ENGAGING HEMOSTASIS DEVICE

(75) Inventor: Gregory J. Bakos, Mason, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: ETHICON ENDO-SURGERY, INC., Cincinnati, OH (US)

(21) Appl. No.: 11/681,435

(22) Filed: Mar. 2, 2007

(51) Int. Cl. A61B 18/14

(52) U.S. Cl. 606/49

(57) ABSTRACT

Various methods and devices are provided for cauterizing tissue. In one embodiment, a device for cauterizing tissue is provided and includes an elongate shaft adapted to be inserted through a body lumen, and a bipolar hemostasis probe disposable through the flexible elongate shaft and having a distal end adapted to cauterize tissue. An articulating jaw can be movably coupled to a distal end of the elongate shaft such that the articulating jaw and the distal end of the probe are adapted to grasp tissue therebetween. The probe can have at least one electrode disposed on a distal end thereof, and a proximal end of the probe can be adapted to couple to an energy source for delivering energy to the distal end of the probe to facilitate cauterization of tissue.

### BACKGROUND

Various types of probes are commonly used in a wide variety of medical procedures, for example, during gastrointestinal procedures, to treat a bleeding site. The probes often include an electrode disposed on its tip to deliver energy to the tissue for cauterization. These probes are positioned with the electrode at the effective tissue site to allow the electrode to cauterize the tissue.

While these devices are effective at treating tissue to stop bleeding, they can be imprecise and potentially cause damage. This can occur because the electrode located at the tip of the probe is simply placed against the tissue to be cauterized. There is no control over the precise location of the electrode against the tissue, and there is also no ability to secure the position of the electrode once tissue cauterization has begin. This typically causes cauterization of a larger area of tissue than is actually necessary.

Accordingly, there is a need for improved cauterization devices having increased precision with which a probe can be used to cauterize tissue.

### SUMMARY

The present invention provides various methods and devices for cauterizing tissue. In one embodiment, a device for cauterizing tissue is provided and includes a flexible elongate shaft adapted to be inserted through a body lumen, and a bipolar hemostasis probe disposable through the flexible elongate shaft and having a distal end adapted to cauterize tissue. An articulating jaw can be movably coupled to a distal end of the flexible elongate shaft such that the articulating jaw and the distal end of the probe are adapted to grasp tissue therebetween. In an exemplary embodiment, the probe has at least one electrode disposed on a distal end thereof. The electrode can be configured in a variety of ways, including being coiled around a portion of the probe or extending longitudinally along a portion of the probe. A proximal end of the probe can be adapted to couple to an energy source for delivering energy to the electrode to cauterize tissue.

The flexible elongate shaft can have a variety of configurations, but in one embodiment disposed through the first lumen of the flexible elongate shaft. An articulating mechanism, such as one or more cables, can extend through the second lumen of the flexible elongate shaft and can be coupled to the articulating jaw such that the articulating mechanism is adapted to effect movement of the articulating jaw. The articulating mechanism can be effective to move the articulating jaw between a first position in which the articulating jaw is spaced apart from the distal end of the probe, and second position in which the articulating jaw and the distal end of the probe are adapted to grasp tissue therebetween. A proximal end of the articulating mechanism can be coupled to an actuator disposed on a handle that is coupled to a proximal end of the flexible elongate shaft, and it can be effective to axially move the articulating mechanism to effect movement of the articulating jaw. In one embodiment, the articulating jaw can be adapted to apply a predetermined force to tissue engaged between the probe and the articulating jaw.

Methods for cauterizing tissue are also provided, and in one embodiment the method can include inserting a flexible elongate shaft through a body lumen, and positioning a distal end of a probe extending from a distal end of the flexible elongate shaft in contact with tissue to be cauterized. A jaw coupled to the distal end of the flexible elongate shaft can be articulated to grasp the tissue between the jaw and the distal end of the probe, and energy can be delivered to the distal end of the probe to cauterize the tissue. The jaw can be articulated by, for example, axially moving an articulating mechanism extending through the elongate shaft and coupled to the jaw. The method can also include applying a predetermined force, for example, in the range of 50 to 100 grams, to tissue grasped between the distal end of the probe and the jaw. In one embodiment, the distal end of the probe is positioned within the stomach.

### BRIEF DESCRIPTION OF THE DRAWINGS

The invention will be more fully understood from the following detailed description taken in conjunction with the accompanying drawings, in which:

FIG. 1 is a partially cross-sectional view of one exemplary embodiment of a device for cauterizing tissue, showing an articulating jaw in a first, open position;

FIG. 2 is a partially cross-sectional view of the device of FIG. 1, showing the articulating jaw in a second, tissue-engaging position;

FIG. 3 is a cross-sectional view of a shaft of the device of FIG. 1;

FIG. 4 is a side view of one exemplary embodiment of a probe of the device of FIG. 1;

FIG. 5 is a side view of another exemplary embodiment of a probe of the device of FIG. 1; and

FIG. 6 is a cross-sectional view of the probe of FIG. 4 disposed within a lumen of the shaft of FIG. 3.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

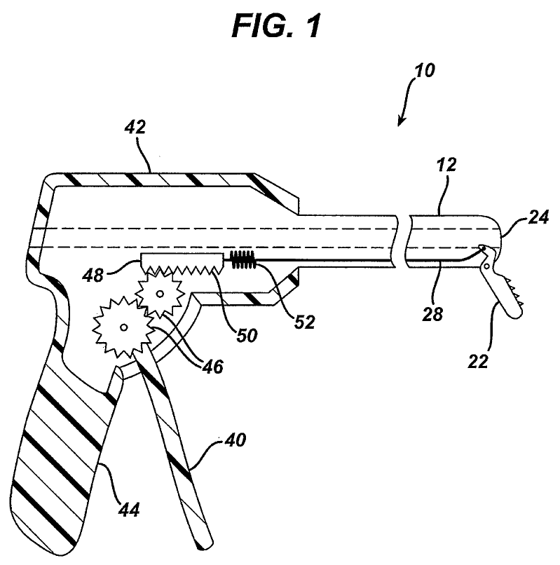

Various exemplary methods and devices are provided for cauterizing tissue. FIG. 1 illustrates one exemplary embodiment of a device 10 for cauterizing tissue. As shown, the device 10 generally includes a handle 42 with an elongate shaft 12 extending therefrom and configured to be introduced translumenally, e.g., through a body lumen. A probe 18, shown in FIG. 4, is disposable through the elongate shaft 12 and has a distal end 20 that is adapted to cauterize tissue. A jaw 22 is movably coupled to a distal end 24 of the elongate shaft 12 and it is configured to articulate such that the jaw 22 and the probe 18 can grasp tissue therebetween. The device can also include an articulating mechanism 28 disposed through or extending along the elongate shaft 12 and coupled to the jaw 22.

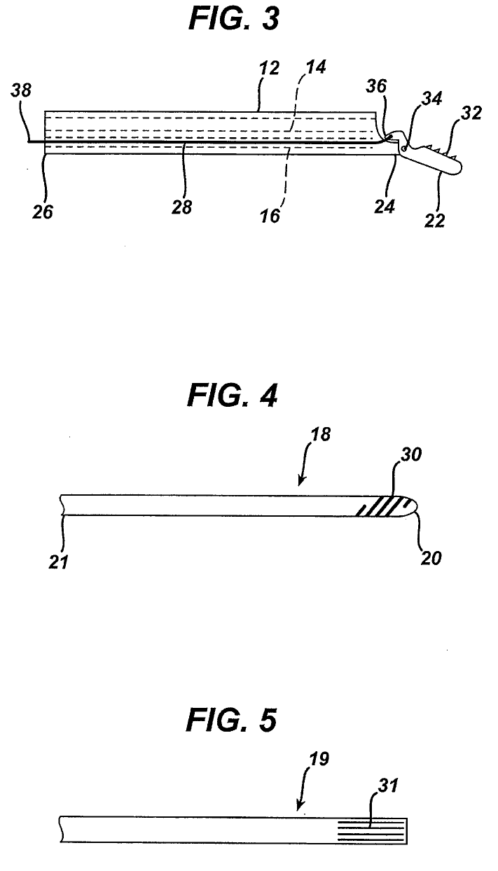

The elongate shaft 12 of the device 10 can have a variety of configurations, and it can be flexible or rigid depending on the intended use. In an exemplary embodiment, the shaft 12, shown in FIG. 3, can be adapted to be inserted translumenally, and therefore at least portions of the shaft 12 can be semi-flexible or flexible to allow insertion through a tortuous lumen. One skilled in the art will appreciate that the shaft 12 can be made from a variety of biocompatible materials that have properties sufficient to enable the shaft 12 to be inserted and moved within channels of a body lumen. The shaft 12 can also have an elongate length to allow the distal end 24 of the shaft 12 to be positioned within the body while a proximal end 26 remains external to the body. As shown in FIG. 3, the shaft 12 can also include first and second lumens 14, 16 extending therethrough for receiving the probe 18 and an articulating mechanism 28. A person skilled in the art will appreciate that the lumens 14, 16 of the shaft 12 can also be used to pass other instruments or fluids through the device 10 for use during a surgical procedure, and that additional lumens can extend through the elongate shaft 12 for this purpose. While only two lumens are shown, the shaft 12 can have only one lumen or any number of lumens.

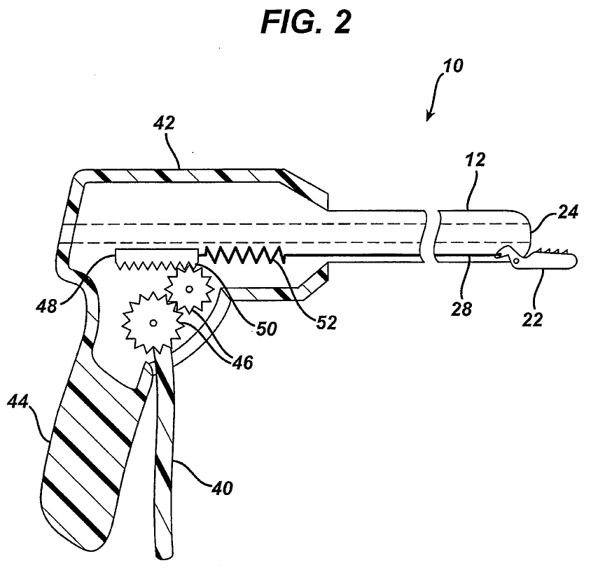

The probe 18 can also have a variety of configurations, but in an exemplary embodiment it is disposable through one of the lumens, e.g., the first lumen 14, of the elongate shaft 12. The probe 18 can be slidable through the first lumen 14, or can be fixed and positioned within the first lumen 14 to allow a distal end 20 of the probe 18 to cauterize tissue. In other embodiments, the probe 18 can be integrally formed with the elongate shaft 12. As the probe 18 is preferably adapted to cauterize tissue, the distal end 20 of the probe 18 can include an electrode 30 formed on or coupled thereto. The electrode 30 can be made from a variety of conductive materials that have properties sufficient to enable the electrode 30 to conduct and deliver energy to tissue. Exemplary conductive materials include, by way of non-limiting example, stainless steel, nitinol, carbon steel, aluminum, and combinations thereof. The electrode 30 can also have a variety of shapes and sizes. For example, the electrode 30 can be coiled around a portion of the distal end 20 of the probe 18 as shown in FIG. 4, or an electrode 31 can extend longitudinally along a portion of a probe 19, as shown in FIG. 5. A person skilled in the art will appreciate that the electrode 30 can have any configuration to facilitate cauterization of tissue. The probe 18 is also adapted to deliver energy to the electrode 30 on the distal end 20 of the probe 18. The energy source can be an internal energy source, such as a battery disposed in the handle 42, or an external energy source. The device 10 can also include an actuating element, shown in FIGS. 1-2, to enable the delivery of energy from an energy source to the electrode 30. By way of non-limiting example, the actuating element can be a button, a switch, a knob, or any other member configured to actuate delivery of energy from an energy source through the probe 18 and to the electrode 30. One skilled in the art will appreciate that the actuating element can be located elsewhere, including on a foot pedal.

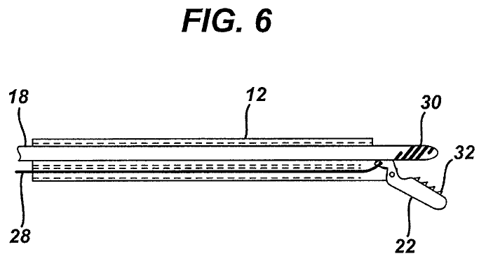

As previously indicated, the elongate shaft 12 can also include a jaw 22, shown in FIGS. 3 and 6, that is movably coupled to the distal end 24 of the elongate shaft 12. The jaw 22 is adapted to move between a first position in which the jaw 22 is spaced apart from the probe 18 for receiving tissue therebetween, and a second position in which the jaw 22 and the distal end 20 of the probe 18 are adapted to grasp tissue therebetween. The jaw 22 can have various configurations, but in the illustrated embodiment the jaw 22 has a generally elongate shape that extends distally from the distal end 24 of the elongate shaft 12. A proximal end of the jaw 22 is pivotally coupled to a distal end 24 of the shaft 12 to allow the jaw 22 to move between the first and second positions. The jaw 22 can also include tissue-engaging features disposed thereon and adapted to engage tissue when the jaw 22 is in the second position. In the illustrated embodiment, the jaw 22 includes a plurality of teeth 32 formed along a surface of the jaw 22.

In order to move the jaw 22 between the first and second positions, the device 10 can include an articulating mechanism 28. In the illustrated embodiment, the articulating mechanism 28 is in the form of a cable that extends through the second lumen 16 of the flexible elongate shaft 12 and that is coupled at a distal end 36 to the jaw 22. A proximal end 38 of the articulating mechanism 28 can be coupled to a control member, which will be discussed in more detail below, for controlling movement of the jaw 22 between the first and second positions. A person skilled in the art will appreciate that the articulating mechanism 28 can have any form that facilitates the movement of the jaw 22. The articulating mechanism 28 can also be configured to apply a predetermined force to the tissue grasped between the distal end 20 of the probe 18 and the jaw 22. In one embodiment, the force can be determined using a spring coupled to the control member and the articulating mechanism 28, as discussed in more detail below. For example, the force can be in the range of 15 to 150 grams, and preferably in the range of 50 to 100 grams.

As previously discussed, the elongate shaft 12 extends from the handle 42 which can have any shape and size, but is preferably adapted to facilitate grasping and manipulation of the device 10. In the illustrated embodiment, as shown in FIGS. 1-2, the handle 42 includes a gripping portion 44 for a user to hold the handle 42 of the device 10. The handle also includes a control member, as indicated above, that can have a variety of configurations to allow control of the articulating mechanism 28 to move the jaw 22 between the first and second positions. In the illustrated embodiment shown in FIGS. 1-2, the control member is in the form of a trigger 40 disposed on the handle 42 coupled to the proximal end 26 of the flexible elongate shaft 12. The trigger 40 is coupled to a series of gears 46 which are coupled to a rack 48 having a plurality of teeth 50 along its length. Movement of the trigger 40 causes the gears 46 to engage the teeth 50 of the rack 48 to move the rack 48 in a proximal direction. This proximal movement of the rack 48 causes the rack 48 to pull on a spring 52 coupled to a distal end of the rack 48 and to the proximal end of the articulating mechanism 28. The spring 52 can be used to configure the articulating mechanism 28 to apply a predetermined force to tissue, as discussed above, based on the tension of the spring 52. For example, a tightly-wound spring will yield a higher force than a loosely-wound spring. As shown in FIGS. 1-2, pulling the spring 52 in a proximal direction causes the spring 52 to pull on the articulating mechanism 28, thereby moving the jaw 22 from the first position into the second position to cause the probe 18 and the jaw 22 to grasp tissue therebetween. A person skilled in the art will appreciate that a variety of other techniques can be used to move the jaw 22 and/or to apply a predetermined force to tissue grasped between the jaw 22 and the probe 18.

The present invention also provides methods for cauterizing tissue. In one exemplary embodiment, the device 10 can be inserted translumenally, e.g., through a natural orifice, and positioned within a body lumen. The device 10 is then positioned adjacent to tissue to be cauterized. The probe 18 can be inserted into the first lumen 14 of the flexible elongate shaft 12 before or after the device is inserted translumenally to position the electrode 30 disposed on the distal end 20 of the probe 18 adjacent to the tissue. The probe 18 can also be pre-disposed through the elongate shaft 12 and positioned so the electrode 30 extends from the distal end 24 of the elongate shaft 12. In order to grasp the tissue, the jaw 22 can be moved from the first position as shown in FIG. 1, to the second position as shown in FIG. 2. The control member, e.g., the trigger 40, can be used to cause the articulating mechanism 28 to move the jaw 22 into the second position, as previously explained. When in the second position, the tissue is grasped between the distal end 20 of the probe 18 and the jaw 22. Energy can be delivered through the probe 18 to the electrode 30 disposed on the distal end thereof to cauterize the tissue held between the probe 18 and the jaw 22. When the cauterization is complete, the control member 40 can be released, causing the jaw 22 to move back into the first position and release the tissue.

In one exemplary embodiment, the device 10 is inserted and positioned within the stomach. The distal end 20 of the probe 18 can be positioned against a wall of the stomach. The control member 40 can be used to cause the articulating mechanism 28 to move the jaw 22 into the second position to grasp a portion of the stomach wall between the distal end 20 of the probe 18 and the jaw 22. This allows for cauterization of tissue within the stomach.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device for cauterizing tissue, comprising:
a flexible elongate shaft adapted to be inserted through a body lumen;
a bipolar hemostasis probe disposable through the flexible elongate shaft and having a distal end adapted to cauterize tissue; and
an articulating jaw movably coupled to a distal end of the flexible elongate shaft such that the articulating jaw and the distal end of the bipolar hemostasis probe are adapted to grasp tissue therebetween.

2. The device of claim 1, wherein the bipolar hemostasis probe has at least one electrode disposed on a distal end thereof.

3. The device of claim 2, wherein the electrode is coiled around a portion of the bipolar hemostasis probe.

4. The device of claim 2, wherein the electrode extends longitudinally along a portion of the bipolar hemostasis probe.

5. The device of claim 1, wherein the flexible elongate shaft has first and second lumens extending therethrough.

6. The device of claim 5, wherein the bipolar hemostasis probe is slidably disposed through the first lumen of the flexible elongate shaft, and an articulating mechanism extends through the second lumen of the flexible elongate shaft and is coupled to the articulating jaw such that the articulating mechanism is adapted to effect movement of the articulating jaw.

7. The device of claim 6, wherein the articulating mechanism comprises at least one cable.

8. The device of claim 6, wherein the articulating mechanism is effective to move the articulating jaw between a first position in which the articulating jaw is spaced apart from the distal end of the bipolar hemostasis probe, and second position in which the articulating jaw and the distal end of the bipolar hemostasis probe are adapted to grasp tissue therebetween.

9. The device of claim 6, wherein a proximal end of the articulating mechanism is coupled to an actuator disposed on a handle that is coupled to a proximal end of the flexible elongate shaft, the actuator being effective to axially move the articulating mechanism to effect movement of the articulating jaw.

10. The device of claim 9, wherein the articulating jaw is adapted to apply a predetermined force to tissue engaged between the probe and the articulating jaw.

11. The device of claim 1, wherein a proximal end of the bipolar hemostasis probe is adapted to couple to an energy source for delivering energy to the distal end of the probe to facilitate cauterization of tissue.

12. A device for cauterizing tissue, comprising:
a flexible elongate shaft having proximal and distal ends;
a jaw pivotally coupled to the distal end of the flexible elongate shaft; and
an electrode extending distally from the distal end of the flexible elongate shaft such that the jaw and the electrode are adapted to engage tissue therebetween.

13. The device of claim 12, wherein the electrode is disposed on a distal end of a probe.

14. The device of claim 13, wherein the probe is disposed through a lumen extending through the elongate shaft, the distal end of the probe adapted to extend distally from the lumen.

15. The device of claim 16, wherein the probe is slidably disposed through the lumen.

16. The device of claim 12, further comprising an articulating mechanism extending through the elongate shaft and being effective to move the jaw between first and second positions.

17. The device of claim 16, wherein the jaw includes tissue-engaging features disposed thereon and adapted to engage tissue when the jaw is in the second position.

18. The device of claim 16, wherein the jaw extends substantially parallel to the flexible elongate member in the first position, and wherein the jaw is angled toward the probe in the second position.

19. The device of claim 16, wherein a proximal end of the articulating mechanism is coupled to a control member that is adapted to control movement of the articulating jaw between the first and second positions.

20. The device of claim 19, further comprising a handle coupled to a proximal end of the flexible elongate shaft, the control member being disposed on the handle.

21. The device of claim 20, wherein the control member is adapted to apply a substantially constant force to the jaw when the jaw is in the second position.

22. The device of claim 16, wherein the articulating mechanism comprises at least one cable with a proximal end coupled to a handle and a distal end coupled to the jaw.

23. A method of cauterizing tissue, comprising:
inserting a flexible elongate shaft through a body lumen;
positioning a distal end of a probe extending from a distal end of the flexible elongate shaft in contact with tissue to be cauterized;
articulating a jaw coupled to the distal end of the flexible elongate shaft to grasp the tissue between the jaw and the distal end of the probe; and
delivering energy to the distal end of the probe to cauterize the tissue.

24. The method of claim 23, wherein articulating the jaw comprises axially moving an articulating mechanism extending through the elongate shaft and coupled to the jaw.

25. The method of claim 23, further comprising applying a predetermined force to tissue grasped between the distal end of the probe and the jaw.

26. The method of claim 25, wherein the predetermined force is in the range of 50 to 100 grams.

27. The method of claim 23, wherein the distal end of the probe is positioned within the stomach.
